(19) United States
(12) Patent Application Publication
(10) Pub. No.: US 2020/0016027 A1
(43) Pub. Date: Jan. 16, 2020

(54) APPARATUS FOR PROVIDING MASSAGE AND METHOD FOR CONTROLLING APPARATUS FOR PROVIDING MASSAGE

(71) Applicant: LG ELECTRONICS INC., Seoul (KR)

(72) Inventors: Sang Won KIM, Seoul (KR); Sun Chan PARK, Seoul (KR)

(73) Assignee: LG ELECTRONICS INC., Seoul (KR)

(21) Appl. No.: 16/561,434

(22) Filed: Sep. 5, 2019

(30) Foreign Application Priority Data
Jun. 28, 2019 (KR) 10-2019-0078022

(51) Int. Cl. A61H 15/00; G06N 20/00; G06F 3/01; G06N 3/08

(52) U.S. Cl. CPC A61H 15/0078; G06N 20/00; G06F 3/011; G06N 3/084; A61B 5/6891; A61H 2201/5046; A61H 2201/5035; A61H 2201/5048; A61H 2201/0149

(57) ABSTRACT

A massage providing apparatus operated by executing an artificial intelligence algorithm and/or machine learning algorithm in a 5G environment connected for the Interne of things and a method for controlling the massage providing apparatus. A method for controlling a massage providing apparatus which may include acquiring information related to image content from an image content playback device for playing back arbitrary image content, analyzing the information related to the image content, setting a massage mode according to a result of analyzing the information related to the image content, and controlling the massage providing apparatus to operate according to the set massage mode.

### CROSS-REFERENCE TO RELATED APPLICATION

This present application claims benefit of priority to Korean Patent Application No. 10-2019-0078022, entitled “APPARATUS FOR PROVIDING MASSAGE AND METHOD FOR CONTROLLING APPARATUS FOR PROVIDING MASSAGE,” filed on Jun. 28, 2019, in the Korean Intellectual Property Office, the entire disclosure of which is incorporated herein by reference.

### BACKGROUND

### 1. Technical Field

The present disclosure relates to a massage providing apparatus and a method for controlling the massage providing apparatus, and more particularly, to a massage providing apparatus for automatically setting and controlling operation of the massage providing apparatus on the basis of information related to image content of the massage providing apparatus capable of interworking with an image content playback device, and a method for controlling the massage providing apparatus.

### 2. Description of Related Art

Massage chairs provide a massage to a user by using a special mechanism to relax stiff muscles or relieve fatigue and stress. Typical massage chairs are configured so that massage rollers or massage protrusions are arranged at positions corresponding to the neck, arms, back, waist, bottom, legs, etc. of a user, and the massage rollers or massage protrusions are controlled according to operation of a motor so as to provide a massage to the user.

Furthermore, massage chairs may be configured not only as a typical chair type but also as a sitting mat type so as to be movable, or may be configured as a car seat in which a massage mechanism is embedded.

Recently, with the development and propagation of services related to virtual reality (VR), researches about a technology for associating electronic products, vehicles, and the like with VR to combine utilization of VR content are being actively carried out.

In particular, related art 1 and related art 2 disclose technologies for controlling massage chairs using VR content.

Related art 1 discloses a technology for executing a massage program and VR content automatically determined on the basis of a result of analyzing brainwave information about a user. However, since only the brainwave information about a user is considered to determine the VR content and massage program, related art 1 is limited in terms of motion control optimized for image content and user optimization according to user preferences.

Related art 2 discloses a technology for providing a virtual reality image and controlling a massage chair according to the contents of the virtual reality image when a user uses the massage chair. However, since only background information, audio information, and object information about the virtual reality image are considered to determine an ambience of the virtual reality image and control the massage chair, related art 2 is limited in terms of user-customized control, and thus may be contrary to a user's intention to take a rest by receiving a massage.

The above-described background art is technical information retained by the inventor to derive the present invention or acquired by the inventor while deriving the present invention, and thus should not be construed as publicly known art that was known prior to the filing date of the present invention.

### SUMMARY OF THE INVENTION

An aspect of the present disclosure is to solve the problem of the related art in which a motion control optimized for image content cannot be performed, and a massage mode optimized for a user cannot be provided since only one piece of information is considered in order to set a massage mode of a massage providing apparatus capable of interworking with an image content playback device.

An aspect of the present disclosure is to solve the problem of the related art in which a massage mode that is contrary to a user's intention to take a rest by receiving a massage is provided since only background information, audio information, and object information about a virtual reality image are considered in order to determine an ambience of the virtual reality image and control a massage chair.

An aspect of the present disclosure is to automatically set and operate a massage mode of a massage providing apparatus on the basis of information related to image content of an image content playback device so as to improve user's satisfaction with the use of the massage providing apparatus and relieve a sense of boredom experienced by the user while using the massage providing apparatus.

An aspect of the present disclosure is to improve user's satisfaction and maximize the effect of a fun function by changing detailed options of a massage mode on the basis of object movement information including at least one among a movement amount of an object included in image content and a movement direction of the object and performing a motion control optimized for the image content.

An aspect of the present disclosure is to provide a further optimized massage mode to a user to improve user's satisfaction with the use of a massage providing apparatus by recommending and setting a massage mode in which user's preference is reflected on the basis of first history information based on information related to image content and second history information based on a manual operation signal from the user.

An aspect of the present disclosure is to improve user's satisfaction by recommending a user-customized massage mode on the basis of information related to image content, user preference, and weather information.

An aspect of the present disclosure is to improve user's satisfaction by recommending a user-customized massage mode on the basis of information related to image content, user preference, weather information, and health information.

An aspect of the present disclosure is to solve the problem of the related art in which a separate charging unit is necessary for charging an image content playback device, by supporting a function of charging the image content playback device in a massage providing apparatus.

An aspect of the present disclosure is to improve user's satisfaction by visually providing a sense of being massaged by synthesizing and outputting image content including a virtual massaging object to a real world on the basis of mixed reality (MR) image content.

An aspect of the present disclosure is to relieve a sense of boredom experienced by a user during a massage and meet the original purpose of receiving a massage and taking a rest by synthesizing and outputting image content including a virtual massaging object to a real world on the basis of mixed reality (MR) image content and by providing a function in which the virtual objects acts or dialogizes in response to a speech of the user.

An aspect of the present disclosure is to automatically set and operate a massage mode of a massage providing apparatus on the basis of at least one among information related to image content, user preference, travel information, traffic information, health information, and weather information in an autonomous vehicle, so as to improve user's satisfaction with the use of the massage providing apparatus and improve the usability of the massage providing apparatus.

An aspect of the present disclosure is not limited to the above-mentioned aspects, and other aspects and advantages of the present disclosure, which are not mentioned, will be understood through the following description, and will become apparent from the embodiments of the present disclosure. Furthermore, it will be understood that aspects and advantages of the present disclosure can be achieved by the means set forth in the claims and combinations thereof.

A method for controlling a massage providing apparatus according to an embodiment of the present disclosure may include automatically setting and controlling operation of the massage providing apparatus on the basis of information related to image content from an image content playback device.

In detail, the method for controlling a massage providing apparatus according to an embodiment of the present disclosure may include acquiring information related to image content from an image content playback device for playing back arbitrary image content, analyzing the information related to the image content, setting a massage mode according to a result of analyzing the information related to the image content, and controlling the massage providing apparatus to operate according to the set massage mode.

According to the method for controlling a massage providing apparatus according to an embodiment of the present disclosure, a massage mode of the massage providing apparatus may be automatically set and operated on the basis of information related to image content of the image content playback device so as to improve user's satisfaction with the use of the massage providing apparatus.

Furthermore, the analyzing may include analyzing the type of the image content from the information related to the image content, and analyzing object movement information including at least one among a movement amount of an object included in the image content and a movement direction of the object from the information related to the image content.

Through the analyzing according to an embodiment of the present disclosure, the massage mode of the massage providing apparatus may be more specifically set according to the type of the image content, the movement amount of the object included in the image content, and the movement direction of the object from the information related to the image content so as to provide the massage mode optimized for the image content.

Furthermore, the setting may include setting a massage course included in the massage mode according to the type of the image content.

Furthermore, the setting may include setting the massage course including at least one massage portion among a foot, leg, arm, waist, back, shoulder, neck, head, and whole body of the user sitting on the massage providing apparatus.

Furthermore, the setting may include setting a massage option included in the massage mode according to the movement information about an arbitrary object included in the image content.

Furthermore, the setting may include setting the massage option including at least one among a massage strength of the massage providing apparatus, a massage position change direction, a massage direction in a corresponding massage portion, and a movement direction of the massage providing apparatus.

Through the setting according to an embodiment of the present disclosure, an optimal motion control is performed by setting the massage course according to the type of the image content and setting the massage option included in the massage mode according to the movement information about the arbitrary object included in the image content, and thus a sense of boredom experienced by the user may be relieved when using the massage providing apparatus, and the effect of a fun function may be maximized.

The method for controlling the massage providing apparatus may further include acquiring at least one among first history information including the massage mode set by using at least one among the type of image content and object movement information and second history information including the massage mode set by receiving a manual operation signal from a user, analyzing a preference of the user using the first history information and the second history information, and setting the massage mode according to a result of analyzing the preference of the user.

Furthermore, the acquiring the at least one may further include acquiring at least one among a cumulative usage amount, a cumulative usage period, and a cumulative number of times of operation change with regard to the massage modes included in the first history information and the second history information.

According to the method for controlling a massage providing apparatus according to an embodiment of the present disclosure, a further optimized massage mode may be provided to the user to improve user's satisfaction with the use of the massage providing apparatus by recommending and setting the massage mode in which user's preference is reflected on the basis of the first history information based on the information related to image content and the second history information based on a manual operation signal from the user.

Furthermore, the acquiring the information may include acquiring the information related to the image content from the image content playback device which plays back one among augmented reality (AR) image content, virtual reality (VR) image content, and mixed reality (MR) image content.

Through the acquiring according to an embodiment of the present disclosure, image content including a virtual massaging object is synthesized and output to a real world on the basis of not only augmented reality (AR) image content and virtual reality (VR) image content but also mixed reality (MR) image content, so that a sense of boredom experienced by the user may be relieved during a massage by visually providing a sense of being massaged, and the user may take a rest while receiving a massage.

A massage providing apparatus according to an embodiment of the present disclosure may include an acquisition unit for acquiring information related to image content from an image content playback device for playing back arbitrary image content, a content analysis unit for analyzing the information related to the image content, a setting unit for setting a massage mode according to a result of analyzing the information related to the image content, and a control unit for controlling the massage providing apparatus to operate according to the set massage mode.

According to the massage providing apparatus according to an embodiment of the present disclosure, a massage mode of the massage providing apparatus may be automatically set and operated on the basis of information related to image content of the image content playback device so as to improve user's satisfaction with the use of the massage providing apparatus.

Furthermore, the content analysis unit may analyze the type of the image content from the information related to the image content, and may analyze object movement information including at least one among a movement amount of an object included in the image content and a movement direction of the object from the information related to the image content.

Through the content analysis unit according to an embodiment of the present disclosure, the massage mode of the massage providing apparatus may be more specifically set according to the type of the image content, the movement amount of the object included in the image content, and the movement direction of the object from the information related to the image content.

Furthermore, the setting unit may set a massage course included in the massage mode according to the type of the image content.

Furthermore, the setting unit may set the massage course including at least one massage portion among a foot, leg, arm, waist, back, shoulder, neck, head, and whole body of the user sitting on the massage providing apparatus.

Furthermore, the setting unit may set a massage option included in the massage mode according to the movement information about an arbitrary object included in the image content.

Furthermore, the setting unit may set the massage option including at least one among a massage strength of the massage providing apparatus, a massage position change direction, a massage direction in a corresponding massage portion, and a movement direction of the massage providing apparatus.

Through the setting unit according to an embodiment of the present disclosure, an optimal motion control is performed by setting the massage course according to the type of the image content and setting the massage option included in the massage mode according to the movement information about the arbitrary object included in the image content, and thus user's satisfaction may be improved, and the effect of a fun function may be maximized.

Furthermore, the massage providing apparatus may further include a history acquisition unit for acquiring at least one among first history information including the massage mode set by using at least one among the type of the image content and the object movement information and second history information including the massage mode set by receiving a manual operation signal from a user, and a preference analysis unit for analyzing a preference of the user using the first history information and the second history information, wherein the setting unit may set the massage mode according to a result of analyzing the preference of the user.

Furthermore, the history acquisition unit may obtain at least one among a cumulative usage amount, a cumulative usage period, and a cumulative number of times of operation change with regard to the massage modes included in the first history information and second history information.

According to the massage providing apparatus according to an embodiment of the present disclosure, a further optimized massage mode may be provided to the user by recommending and setting the massage mode in which user's preference is reflected on the basis of the first history information based on the information related to image content and the second history information based on a manual operation signal from the user.

Furthermore, the acquisition unit may acquire the information related to the image content from the image content playback device which plays back one among augmented reality (AR) image content, virtual reality (VR) image content, and mixed reality (MR) image content.

Through the acquisition unit according to an embodiment of the present disclosure, image content including a virtual massaging object is synthesized and output to a real world on the basis of not only augmented reality (AR) image content and virtual reality (VR) image content but also mixed reality (MR) image content, so that user's satisfaction in terms of rest and fun may be improved by visually providing a sense of being massaged.

Furthermore, the massage providing apparatus may be connected to the image content playback device via at least one among a cradle, a wired cable, and a wireless communication module.

According to the massage providing apparatus according to an embodiment of the present disclosure, convenience of a user may be improved by supporting a function of charging an image content playback device.

In addition, other methods and other systems for implementing the present invention, and computer programs for executing the above method may be further provided.

### BRIEF DESCRIPTION OF THE DRAWINGS

The above and other aspects, features, and advantages of the present disclosure will become apparent from the detailed description of the following aspects in conjunction with the accompanying drawings, in which:

FIG. 1 is an exemplary diagram illustrating a massage providing environment including a massage providing apparatus according to an embodiment of the present disclosure, an output apparatus, a user terminal, a server, and a network for connecting the foregoing elements;

FIG. 2A is a diagram illustrating a state of connection with an image content playback device via a wired cable of the massage providing apparatus according to an embodiment of the present disclosure;

FIG. 2B is a diagram illustrating a state of connection with an image content playback device via a cradle of the massage providing apparatus according to an embodiment of the present disclosure;

FIG. 2C is a diagram illustrating a state of connection with the image content playback device via a wireless communication module of the massage providing apparatus according to an embodiment of the present disclosure;

FIG. 3 is a schematic block diagram illustrating the massage providing apparatus according to an embodiment of the present disclosure;

FIG. 4 is an exemplary diagram illustrating a massage mode control panel displayed on the display unit of the massage providing apparatus of FIG. 3;

FIG. 5 is a schematic block diagram illustrating the processing unit of the massage providing apparatus of FIG. 3;

FIG. 6 is an exemplary diagram for describing operation of the massage providing apparatus according to an embodiment of the present disclosure when mixed reality (MR) image content is played back;

FIG. 7 is an exemplary diagram for describing operation of the massage providing apparatus installed in an autonomous vehicle according to an embodiment of the present disclosure;

FIG. 8 is a flowchart illustrating a method for operating a massage providing apparatus according to an embodiment of the present disclosure; and

FIG. 9 is a flowchart illustrating a method for operating a massage providing apparatus by reflecting a result of analyzing the preference of a user according to an embodiment of the present disclosure.

### DETAILED DESCRIPTION

Advantages and features of the present disclosure and methods for achieving them will become apparent from the descriptions of aspects herein below with reference to the accompanying drawings. However, the present disclosure is not limited to the aspects disclosed herein but may be implemented in various different forms, and should be construed as including all modifications, equivalents, or alternatives that fall within the sprit and scope of the present disclosure. The aspects are provided to make the description of the present disclosure thorough and to fully convey the scope of the present disclosure to those skilled in the art. In relation to describing the present disclosure, when the detailed description of the relevant known technology is determined to unnecessarily obscure the gist of the present disclosure, the detailed description may be omitted.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. Although the terms first, second, etc. may be used herein to describe various elements, these elements should not be limited by these terms. These terms may be only used to distinguish one element from other elements.

Hereinafter, embodiments of the present disclosure will be described in detail with reference to the accompanying drawings. Like reference numerals designate like elements throughout the specification, and overlapping descriptions of the elements will not be provided.

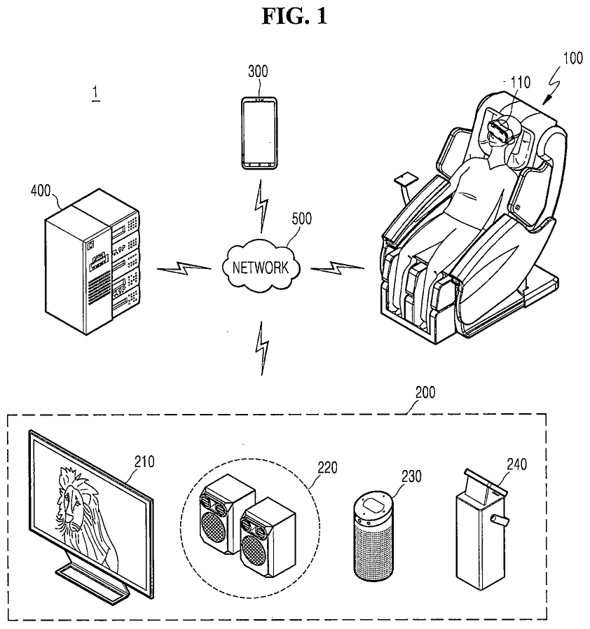

FIG. 1 is an exemplary diagram illustrating a massage providing environment including a massage providing apparatus according to an embodiment of the present disclosure, an output apparatus, a user terminal, a server, and a network for connecting the foregoing elements. Referring to FIG. 1, a massage providing environment 1 may include a massage providing apparatus 100, an output apparatus 200, a user terminal 300, a server 400, and a network 500.

The massage providing apparatus 100 may include a small tool for manually providing a massage, a partial massager for massaging arms, legs, neck, or the like, which are parts of a human body, and a massage seat and massage chair for massaging an entire body.

In the present embodiment, the massage providing apparatus 100 is exemplarily configured as a massage chair, and such the massage providing apparatus 100 may also be installed in an autonomous vehicle.

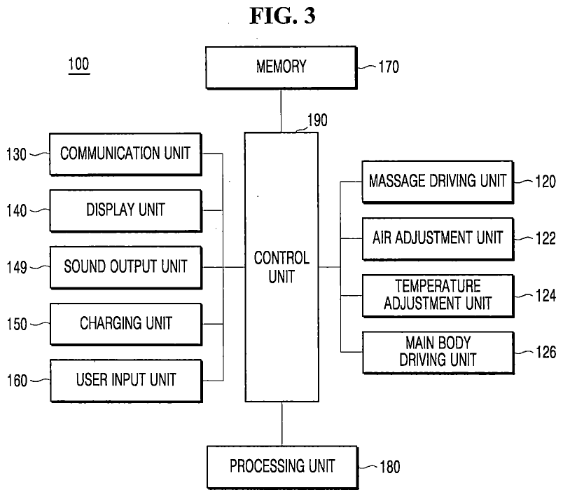

Furthermore, the massage providing apparatus 100 may be divided into a seat (not shown) that contacts a human body when a user is sitting and a driving unit (120 of FIG. 3) disposed in the seat to operate to provide a massage to the user.

The seat is divided into an upper end part, a seating part, and a lower end part according to locations on which a human body is placed. That is, the seat may include the upper end part having a shape surrounding a head of the user, the seating part having a shape on which the user can sit with its upper body leaning thereon, and the lower end part having a shape surrounding calves and feet of the user, so as to give a sense of comfort and relaxedness to the user. Here, a size and position of each part of the seat may be changed manually or automatically according to body characteristics of the user. Furthermore, the driving unit, which includes an actuator for providing a massage service, may massage an entire body of the user while varying positions and strengths according to a set massage mode by operating a massage roll or injecting air.

Furthermore, the massage providing apparatus 100 may include a sensor unit (not shown) to obtain body information about the user sitting on the massage providing apparatus 100. For example, the massage providing apparatus 100 may obtain height, weight, and skeletal position information, etc. about the user via the sensor unit. Furthermore, the massage providing apparatus 100 may detect a fingerprint to obtain registration information about the user, and may provide a service corresponding to the user.

In the present embodiment, the massage providing apparatus 100 may interwork with an image content playback device 110 to obtain, from the image content playback device 110 playing back arbitrary image content, and analyze information related to the image content, and set a massage mode according to a result of analyzing the information related to the image content, and the massage providing apparatus 100 may be controlled so as to operate according to the set massage mode.

Here, the image content playback device 110 may include a head mounted display (HMD), which is an image display device that may be worn on the head of the user like glasses to allow the user to enjoy images. However, a type of the image content playback device 110 is not limited, and may include any wearable-type display device. Furthermore, in the present embodiment, the image content playback device 110 may provide, to the massage providing apparatus 100, the information related to image content being played back. Here, the image content playback device 110 is only capable of displaying and sensing, and image content to be played back by the image content playback device 110 may be selected via the massage providing apparatus 100 and the user terminal 300. That is, the image content playback device 110 interworking with the massage providing apparatus 100 may only serve to play back image content and perform sensing for inputting user information. However, an embodiment of the present disclosure is not limited thereto, and thus the image content playback device 110 or the server 400 may analyze the information related to image content to provide the analyzed information to the massage providing apparatus 100. Furthermore, the image content playback device 110 may play back one among augmented reality (AR) image content, virtual reality (VR) image content, and mixed reality (MR) image content.

Furthermore, the massage providing apparatus 100 may obtain at least one among first history information including a massage mode set according to a result of analyzing the information related to image content and second history information including a massage mode set by receiving a manual operation signal from the user, and may analyze a preference of the user using the first history information and the second history information, and may recommend and set a massage mode according to a result of analyzing the preference of the user. That is, the massage providing apparatus 100 may analyze the user preference on the basis of the first history information and the second history information stored in a memory (170 of FIG. 3) to recommend a massage mode.

Here, the term “content” may generally represent individual information elements or digital information including characters, symbols, voices, sounds, sound sources, images, moving pictures (video and audio), etc. accessible through the network 500. Such content may include, for example, data such as a text, image, moving picture, sound source, link (e.g., web link), etc. or a combination of at least two pieces of the data. However, in the present embodiment, the content may include image content including a moving picture (video and audio), and the image content playback device 110 may download image content via the network 500, or the user may store image content in the image content playback device 110.

In an optional embodiment, the massage providing apparatus 100 may recommend a massage mode on the basis of weather information. To this end, the massage providing apparatus 100 may collect weather information from a weather server (not shown) for providing weather information, and may communicate with the weather server via the network 500. That is, the massage providing apparatus 100 may combine at least one among the image content analysis result, the user preference, and the weather information for the time of playing back image content so as to recommend and set a massage mode. For example, a massage mode may be differently recommended and set on a cloudy day even for the result of analyzing the same image content.

In the present embodiment, the massage providing apparatus 100 may be disposed in a predetermined space (e.g., a home, company, hospital, or the like), and may search for at least one output apparatus 200, which is disposed in the same space as the massage providing apparatus 100 to output at least one among an audio signal and a video signal, and may adjust an output signal of the detected at least one output apparatus 200 according to an operation mode of the massage providing apparatus 100. Here, the operation mode of the massage providing apparatus 100 may include, for example, a call mode (voice call or video call) interworking with the user terminal 300 or a content playback mode for playing back content. That is, content which is played back in the image content playback device 110 may be allowed to be played back in the output apparatus 200. Furthermore, adjusting the output signal of the output apparatus 200 may include adjusting an output sound volume of an audio signal being output from the output apparatus 200. Furthermore, the operation mode of the massage providing apparatus 100 may include a massage mode.

Furthermore, the massage providing apparatus 100 may select at least one from among at least one output apparatuses 200 detected according to the operation mode, and may transmit at least one among an audio signal and a video signal to the selected output apparatus 200.

Here, selecting the output apparatus 200 may include selecting any one output apparatus 200 for outputting caller information (a video signal, for example, an image of a caller, a name of the caller, a number of the caller, etc.), selecting any one output apparatus 200 for outputting a voice of the caller (an audio signal), or selecting any one output apparatus 200 for outputting the caller information and the voice of the caller during the call mode in which a call between the user and the caller is performed via a display unit (140 of FIG. 3) and a sound output unit (149 of FIG. 3) of the massage providing apparatus 100.

Furthermore, selecting the output apparatus 200 may include selecting any one output apparatus 200 for outputting a video signal included in content, selecting any one output apparatus 200 for outputting an audio signal included in the content, or selecting any one output apparatus 200 for outputting the video signal and audio signal included in the content during the content playback mode in which arbitrary content selected by the user is played back via the content playback device 110 and the sound output unit (149 of FIG. 3) of the massage providing apparatus 100.

This output apparatus 200 may include an electronic device for outputting at least one among an audio signal and a video signal, for example, a TV 210, a two-channel speaker 220, an AI speaker 230, a beam projector 240, an inbuilt speaker (the sound output unit 149 of FIG. 3), or the like. The output apparatus 200 is not limited to the above-mentioned electronic devices in the present embodiment, and thus may include a variety of home appliances (e.g., a washing machine, an air conditioner, a refrigerator, a cleaner, etc.).

At least one among the output apparatuses 200 may receive a control signal including output signal adjustment from the massage providing apparatus 100 to adjust the output sound volume of an audio signal to a volume greater or smaller than a current volume or to an off state.

Furthermore, at least one among the output apparatuses 200 may receive a video signal from the massage providing apparatus 100 and may output the video signal, in response to a video signal output request from the massage providing apparatus 100. Furthermore, at least one among the output apparatuses 200 may receive an audio signal from the massage providing apparatus 100 and may output the audio signal, in response to an audio signal output request from the massage providing apparatus 100. Furthermore, at least one among the output apparatuses 200 may receive a video signal and an audio signal from the massage providing apparatus 100 and may output the video signal and the audio signal, in response to a video signal and audio signal output request from the massage providing apparatus 100.

The user terminal 300 may receive a service for driving or controlling the massage providing apparatus 100 through an authentication process after accessing a massage providing apparatus driving application or massage providing apparatus driving site. In the present embodiment, the massage providing apparatus 100 that has undergone the authentication process may drive the massage providing apparatus 100, and may control operation of the massage providing apparatus 100.

In the present embodiment, the user terminal 300 may be a desktop computer, smartphone, notebook, tablet PC, smart TV, cell phone, personal digital assistant (PDA), laptop, media player, micro server, global positioning system (GPS) device, electronic book terminal, digital broadcast terminal, navigation device, kiosk, MP3 player, digital camera, home appliance, and other mobile or immobile computing devices operated by the user, but is not limited thereto. Furthermore, the user terminal 300 may be a wearable terminal having a communication function and a data processing function, such as a watch, glasses, a hair band, a ring, or the like. The user terminal 300 is not limited to the above-mentioned devices, and thus any terminal that supports web browsing may be used as the user terminal 300. Furthermore, in the present embodiment, the user terminal 300 may be a remote controller or massage bot for controlling operation of the massage providing apparatus 100.

The server 400 may be a database server, which provides big data required for applying a variety of artificial intelligence algorithms and data for operating the massage providing apparatus 100. Furthermore, the server 400 may include a web server or application server for remotely controlling the operation of the massage providing apparatus 100 by using a massage providing apparatus driving application or massage providing apparatus driving web browser installed in the user terminal 300.

Here, the artificial intelligence (AI), which is an area of computer engineering and information technology for studying methods for enabling computers to mimic thinking, learning, self-development, or the like that can be carried out with human intelligence, may represent enabling computers to mimic human intelligent behavior.

Furthermore, the artificial intelligence is directly/indirectly related to other fields of computer science rather than existing per se. In particular, at the present day, researches are being actively carried out to solve problems of various fields of information technology by applying artificial intelligence elements in those fields.

Machine learning, which is an area of the artificial intelligence, may belong to a field of study for imparting to a computer a capability to learn without explicit programs. In detail, the machine learning may be regarded as a technology for constructing systems, which learn and predict on the basis of empirical data and improve their own performance, and algorithms for the systems. Machine learning algorithms may be established so as to construct a specific model for performing prediction or decision on the basis of input data rather than to execute static program instructions that are strictly determined.

The server 400 may receive, from the massage providing apparatus 100, a result of searching for at least one operating output apparatus 200 and the operation mode of the massage providing apparatus 100, and may transmit, to the massage providing apparatus 100, an output signal adjustment control signal of the detected at least one output apparatus 200 according to the operation mode. Furthermore, the server 400 may receive the operation mode of the massage providing apparatus 100 from the massage providing apparatus 100, may transmit a result of selecting at least one from among the at least one output apparatuses 200 to the massage providing apparatus 100, and may control the massage providing apparatus 100 to transmit at least one among an audio signal and a video signal to the selected output apparatus 200.

Furthermore, in the present embodiment, the server 400 may provide image content played back in the image content playback device 110, and may learn a massage mode corresponding to the image content played back in the image content playback device 110 to transmit a learning result to the massage providing apparatus 100. Here, the massage providing apparatus 100 may set the massage mode according to a result of learning of the image content playback device 110. However, the process of learning the massage mode corresponding to the image content played back in the image content playback device 110 may be executed by the massage providing apparatus 100 rather than the server 400.

The network 500 may serve to connect the massage providing apparatus 100, the output apparatus 200, the user terminal 300, and the server 400. The network 500 may include, for example, wired networks such as local area networks (LANs), wide area networks (WANs), metropolitan area networks (MANs), integrated service digital networks (ISDNs), and the like or wireless networks such as wireless LANs, CDMA, Bluetooth, satellite communication, and the like, but the scope of the present disclosure is not limited thereto. Furthermore, the network 500 may transmit/receive information using short-range communications and/or long-distance communications. Here, the short-range communications may include Bluetooth, radio frequency identification (RFID), infrared data association (IrDA), ultra-wideband (UWB), ZigBee, and wireless fidelity (Wi-Fi) technology, and the long-distance communications may include code division multiple access (CDMA), frequency division multiple access (FDMA), time division multiple access (TDMA), orthogonal frequency division multiple access (OFDMA), and single carrier frequency division multiple access (SC-FDMA) technology.

The network 500 may include a connection of network elements such as a hub, a bridge, a router, a switch, and a gateway. The network 500 may include one or more connected networks, for example, a multi-network environment, including a public network such as the Internet and a private network such as a secure corporate private network. Access to the network 500 may be provided via one or more wired or wireless access networks. Furthermore, the network 500 may support the Internet of things (IoT) network for exchanging and processing information between distributed elements such as things or the like and/or 5G communication.

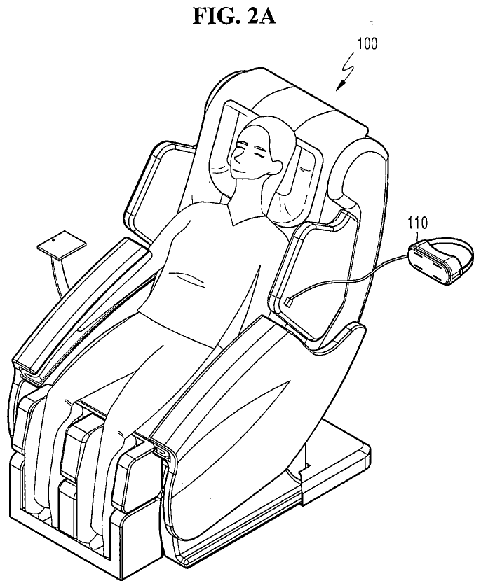

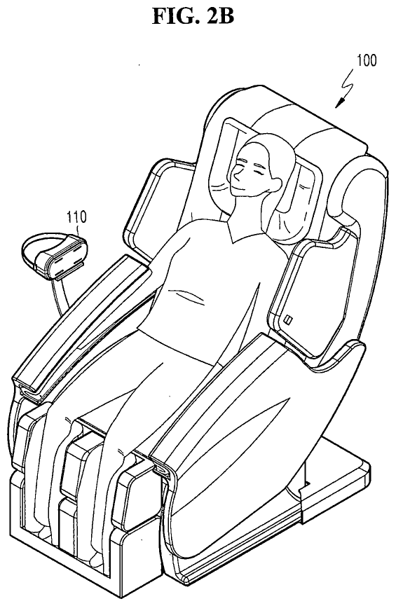

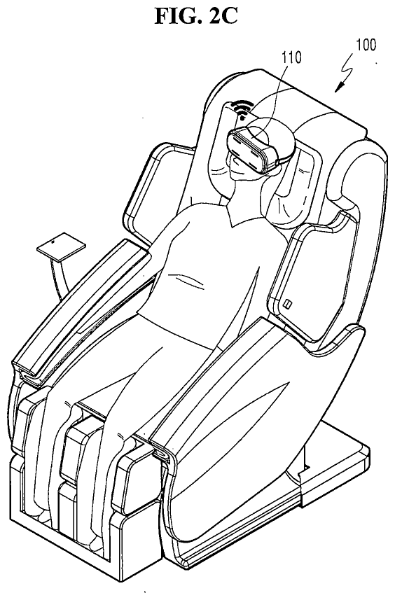

FIGS. 2A to 2C are diagrams schematically illustrating a connection (interwork) structure of a massage providing apparatus according to an embodiment of the present disclosure and an image content playback device. FIG. 2A is a diagram illustrating a state of connection with an image content playback device via a wired cable of the massage providing apparatus according to an embodiment of the present disclosure. FIG. 2B is a diagram illustrating a state of connection with an image content playback device via a cradle of the massage providing apparatus according to an embodiment of the present disclosure. FIG. 2C is a diagram illustrating a state of connection with the image content playback device via a wireless communication module of the massage providing apparatus according to an embodiment of the present disclosure. Descriptions which overlap with the above descriptions related to FIG. 1 are not provided below.

Referring to FIG. 2A, the massage providing apparatus 100 and the image content playback device 110 may be connected via a wired cable so as to transmit/receive data. That is, the massage providing apparatus 100 and the image content playback device 110 may be allowed to interwork with each other by connecting a signal input/output terminal of the massage providing apparatus 100 and a signal input/output terminal of the image content playback device 110 by using a wired cable. Here, the wired cable may represent a line for sending a signal or power, which is achieved by insulating a conductor composed of twisted steel wires or a single wire or by wrapping a common outer cover around the entirety of the conductor. That is, the wired cable may represent a transmission line between the massage providing apparatus 100 and the image content playback device 110. For example, wired networks using the wired cable may include local area networks (LANs), wide area networks (WANs), metropolitan area networks (MANs), integrated service digital networks (ISDNs), and the like.

Furthermore, in the present embodiment, the signal input/output terminal of the massage providing apparatus 100 may be implemented as a charging terminal capable of charging the image content playback device 110. Therefore, it is possible to charge the image content playback device 110 simultaneously by connecting the image content playback device 110 to the massage providing apparatus 100 without separately charging the image content playback device 110.

Furthermore, referring to FIG. 2B, the massage providing apparatus 100 and the image content playback device 110 may be connected in a wireless communication manner via a cradle provided to the massage providing apparatus 100 so as to transmit/receive data. That is, when the user places the image content playback device 110 on the cradle of the massage providing apparatus 100, the massage providing apparatus 100 and the image content playback device 110 may interwork with each other. Therefore, when the image content playback device 110 is placed on the cradle of the massage providing apparatus 100, information related to image content may be acquired from the image content playback device 110. Furthermore, the cradle of the massage providing apparatus 100 may include a wireless charging function, and may perform wireless charging when the image content playback device 110 is placed. This wireless charging may transfer power using at least one wireless power transfer method (e.g., an electromagnetic induction method, an electromagnetic resonance method, an RF wireless power transfer method, etc.).

However, in the present embodiment, the cradle of the massage providing apparatus 100 may perform both a wireless charging function and a cradle function, or may only perform the cradle function. That is, when the cradle of the massage providing apparatus 100 performs the wireless charging function and the cradle function, the massage providing apparatus 100 and the image content playback device 110 may interwork with each other via the wired cable or the wireless communication module described below. Furthermore, when the cradle of the massage providing apparatus 100 only perform the cradle function, the massage providing apparatus 100 and the image content playback device 110 may interwork with each other via the wired cable or the wireless communication module, and the image content playback device 110 may be charged via the wired cable.

Furthermore, referring to FIG. 2C, the massage providing apparatus 100 and the image content playback device 110 may be connected via the wireless communication module so as to transmit/receive data. For example, a wireless network via the wireless communication module may include short-range communications and/or long-distance communications. However, since the massage providing apparatus 100 is generally located at a short distance from the image content playback device 110, and the user uses the massage providing apparatus 100 while wearing the image content playback device 110, descriptions are provided on the assumption that short-range communications is used. Here, the short-range communications may include Bluetooth, radio frequency identification (RFID), infrared data association (IrDA), ultra-wideband (UWB), ZigBee, and wireless fidelity (Wi-Fi) technology.

FIG. 3 is a schematic block diagram illustrating a massage providing apparatus according to an embodiment of the present disclosure. Descriptions which overlap with the above descriptions related to FIGS. 1 and 2 are not provided below. Referring to FIG. 3, the massage providing apparatus 100 may include a communication unit 130, a display unit 140, a sound output unit 149, a charging unit 150, a user input unit 160, a memory 170, a processing unit 180, and a control unit 190.

The communication unit 130 may interwork with the network 500 to provide a communication interface required for providing, in a form of packet data, transmission/reception signals between the massage providing apparatus 100, the user terminal 300, and/or the server 400. Furthermore, the communication unit 130 may support a variety of object-to-object intelligent communication (Internet of things (IoT), Internet of everything (IoE), Internet of small things (IoST), etc.), and may support machine to machine (M2M) communication, vehicle to everything (V2X) communication, device to device (D2D) communication, etc.

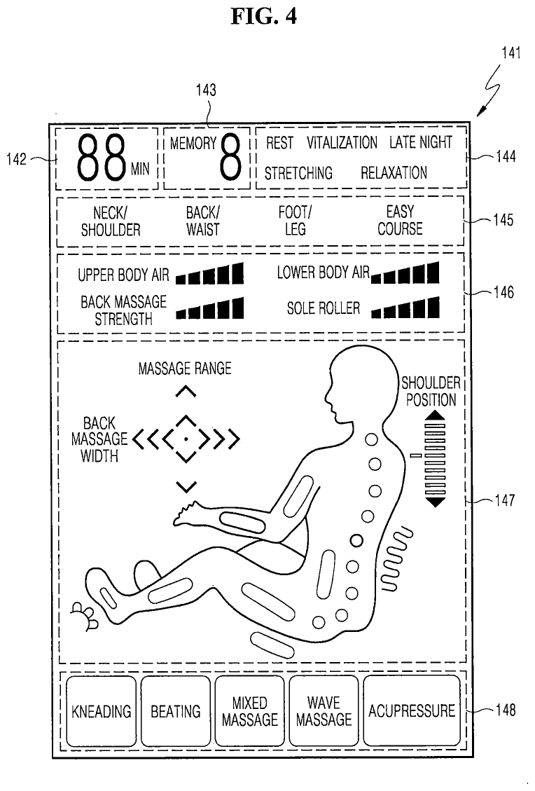

The display unit 140 may display an operation state of the massage providing apparatus 100 under control by the control unit 190. In the present embodiment, the display unit 140 may display a result of setting the massage mode of the massage providing apparatus 100 according to a result of analyzing information related to image content. FIG. 4 illustrates an example of a massage mode control panel 141 displayed on the display unit 140.

Referring to FIG. 4, the massage mode control panel 141 may include an operation time display part 142 for displaying a massage execution time of the massage providing apparatus 100 and a user mode selection part 143 for selecting the massage mode stored in the memory 170. Here, in the present embodiment, an optimal automatic course setting desired by the user may be stored in the memory 170, and when the user selects the massage mode stored in the memory 170, the selected user mode may be displayed on the user mode selection part 143. Furthermore, the massage mode control panel 141 may include automatic massage course selection parts 144 and 145 for selecting an automatically set massage course such as a massage position, strength, etc. The automatic massage course may include, for example, a rest course, a vitalization course, a late-night course, a stretching course, a relaxation course, a neck/shoulder course, a back/waist course, and a leg/foot course. Here, when the user selects a desired course from the courses of the automatic massage course selection parts 144 and 145, the selected massage course may be displayed on the automatic massage course selection parts 144 and 145.

Furthermore, the massage mode control panel 141 may include a massage option adjustment part 146 for adjusting a massage option such as a massage strength or the like, a massage position adjustment part 147 for displaying a current massage performing position and allowing the user to minutely change the position to a desired position, and a manual massage course selection part 148 for selecting a massage operation. Here, the massage option adjustment part 146 may adjust massage options, for example, an upper body air strength, a lower body air strength, a back massage strength, a sole roller massage strength, or the like, and the manual massage course selection part 148 may allow manual selection of a massage course such as kneading, beating, mixed massage, wave massage, acupressure, or the like.

Meanwhile, in the case where the massage mode control panel 141 is configured as a touch panel, the operation time display part 142, the user mode selection part 143, the automatic massage course selection parts 144 and 145, the massage option adjustment part 146, the massage position adjustment part 147, and the manual massage course selection part 148 are configured such that an item corresponding to a selection made in response to a touch input from the user is selected and set, and the selected item is displayed. In particular, the massage position adjustment part 147 outputs a human-like figure so as to assist the user to select a massage position more easily.

Furthermore, the display unit 140 may allow selection of playback image content of the image content playback device 110 interworking with the massage providing apparatus 100, or may display information related to the playback image content. That is, the display unit 140 may display a result of analyzing information related to image content input from the image content playback device 110, and may allow change of a set massage mode according to an analysis result. Furthermore, the display unit 140 may display health-related information such as information about effects depending on massaged portions and massage information suitable for user characteristics such as age, gender, or the like.

In the present embodiment, the display unit 140 may perform not only a function of displaying a variety of information but also a function of an input unit for receiving an input of a predetermined control command so that the user may control overall operation of the massage providing apparatus 100. To this end, the display unit 140 may be configured with a touch-sensitive display controller or other various input/output controllers. For example, the touch-sensitive display controller may provide an output interface and an input interface between an apparatus and a user. The touch-sensitive display controller may transmit/receive electric signals to/from the control unit 190. Furthermore, the touch-sensitive display controller displays a visual output to the user, wherein the visual output may include a text, graphic, image, video, and a combination thereof. Such the display unit 140 may be a predetermined display member such as, for example, a touch-sensitive organic light emitting display (OLED), liquid crystal display (LCD), or light emitting display (LED).

The sound output unit 149 may output all pieces of information displayed on the display unit 140 as an audio signal. Furthermore, the sound output unit 149 may output an audio signal output from the image content playback device 110 interworking with the massage providing apparatus 100, and may output an audio signal (e.g., music playback or the like) from other devices capable of communicating with the massage providing apparatus 100 by wire or wirelessly. Furthermore, in response to control by the control unit 190, the sound output unit 149 may output a warning sound, a notification message pertaining to an operation mode, an operation state, an error state, or the like, information corresponding to a voice command of the user, a processing result corresponding to the voice command of the user, etc. in an audio form.

The sound output unit 149 may convert an electric signal 190 received from the control unit 190 into an audio signal, and may output the audio signal. To this end, the sound output unit 149 may be provided with a speaker (not shown) or the like. Furthermore, in the present embodiment, the massage providing apparatus 100 may further include a sound input unit (not shown). In response to control by the control unit 190, the sound input unit (e.g., a microphone) may receive an input of a speech uttered by the user towards the massage providing apparatus 100. Furthermore, a plurality of microphones may be provided so as to more accurately receive an uttered speech of the user. Here, each of the plurality of microphones may be spaced apart from each other so as to be arranged at different positions, and may process a received uttered speech of the user into an electric signal.

The charging unit 150 may charge the image content playback device 110 via at least one among the wired cable and the cradle capable of wireless charging. Furthermore, the charging unit 150 may provide a charging state of the image content playback device 110 to the control unit 190 so as to output the charging state via the display unit 140 and the sound output unit 149. Here, even when the image content playback device 110 is charged using a separate charging unit, the charging unit 150 may receive an input of the charging state from the image content playback device 110 to provide the charging state to the control unit 190.

The user input unit 160 may represent a sensing unit for sensing user information to provide an optimized massage mode to the user. That is, in the present embodiment, a health condition of the user, an emotional state of the user when the user is being massaged, and the like may be detected and analyzed via the user input unit 160, and an optimized massage mode may be learned and provided to the user by using the detected and analyzed health condition and emotional state. For example, when the user sits on the massage providing apparatus 100, a body shape of the user may be analyzed via the user input unit 160 to recommend a massage mode corresponding to the body shape, and when the user wears the image content playback device 110, a facial expression of the user may be analyzed via the user input unit 160 when the user is being massaged so as to recommend a massage mode for a facial expression classified as a positive expression. Meanwhile, data about the user health condition and user personal information may be directly input by the user using the user terminal 300, the massage mode control panel 141, and voice recognition via the sound input unit.

In more detail, the user input unit 160 may include various sensors (not shown) included in the massage providing apparatus 100 and various sensors (not shown) included in the image content playback device 110 interworking with the massage providing apparatus 100, and sensing data of the user input unit 160 may be combined and analyzed. For example, the sensors provided to the massage providing apparatus 100 may include at least one among a weight detection sensor, an acceleration sensor, a gravity sensor (G-sensor), a gyroscope sensor, a motion sensor, an infrared (IR) sensor, a finger scan sensor, an ultrasonic sensor, an environment sensor (e.g., a barometer, a hygrometer, a thermometer, a radiation detection sensor, a thermal detection sensor, a gas detection sensor, etc.), and a chemical sensor (e.g., an electronic nose, a healthcare sensor, a biometric sensor, etc.).

Furthermore, the sensors provided to the image content playback device 110 may include at least one among a Lidar sensor, an illumination sensor, an acceleration sensor, a gravity sensor, a gyroscope sensor, a motion sensor, an RGB sensor, an infrared sensor, a finger scan sensor, an ultrasonic sensor, an optical sensor, an environment sensor, and a chemical sensor. Meanwhile, in the present embodiment, the massage providing apparatus 100 and the image content playback device 110 may combine and use pieces of information detected by at least two of the above sensors.

The memory 170 may store information that supports various functions of the massage providing apparatus 100. The memory 170 may store a number of applications or application programs executed in the massage providing apparatus 100 and pieces of information and instructions for operating the massage providing apparatus 100. At least a portion of the application programs may be downloaded from an external server via wireless communication. Furthermore, the memory 170 may store user information about at least one user who is to interact with the massage providing apparatus 100. This user information may include face information, body shape information (e.g., image-captured by the user input unit 160), voice information, etc. which may be used to identify a recognized user. Furthermore, the memory 170 may store performance information about the image content playback device 110 interworking with the massage providing apparatus 100. Here, the performance information about the image content playback device 110 may include output strength information, playback image content quality information, and other various pieces of information indicating output performance. For example, the performance information may include a wattage value indicating an output strength, whether a stereo sound is supported, a resolution, and whether 5G communication is supported. Furthermore, the memory 170 may store instructions (control codes) for controlling the image content playback device 110 interworking with the massage providing apparatus 100.

Furthermore, the memory 170 may store various reference values that may be used to analyze information related to image content. For example, the memory 170 may store reference values obtained by digitalizing information related to image content, which may be used to analyze the type of the image content such as multi-play/social, game, racing, sports, horror/mystery, world tour, documentary/history, entertainment, creativity, rest/meditation, experience, music, etc. Here, the information related to image content may include metadata indicating information about the image content and image content identification information. The metadata is secondary information obtained by categorizing information types so that information may be intelligently controlled and structurally accessed. The metadata may represent data including various pieces of information or attributes about lower-level data for describing the lower-level data in an upper level. The metadata is also defined as “data about data” since the metadata determines a data type, and is also referred to as data attribute information since the metadata is given to content according to a fixed rule so as to efficiently search for and use a piece of information being searched for among a large amount of pieces of information.

For example, in the case where movie content is provided, image content metadata may be information about contents related to the movie, such as a movie title, a movie running time, starring actors, a movie type (genre), a movie director, a production company, etc. Furthermore, in the case where TV program content is provided, the image content metadata may be information about contents related to the TV program, such as a program title, a program type (genre), a content file name, cast members, a director, a broadcasting company, etc. Furthermore, the image content identification information may be unique identification information for discriminating metadata of one or more pieces of content and identifying the one or more pieces of content.

The memory 170 may store information (image content metadata and image content identification information) related to analyzed image content. In the memory 170, the information related to image content may be stored in a file form together with image content. Furthermore, the memory 170 may store a variety of data in addition to image content and image content metadata.

In the present embodiment, the memory 170 may perform a function of temporarily or permanently storing data processed by the control unit 190. Here, the memory 170 may include magnetic storage media or flash storage media, but the scope of the present disclosure is not limited thereto. This memory 170 may include an internal memory and/or an external memory, and may include a volatile memory such as a DRAM, SRAM, SDRAM, or the like, a non-volatile memory such as a one time programmable ROM (OTPROM), PROM, EPROM, EEPROM, mask ROM, flash ROM, NAND flash memory, NOR flash memory, or the like, and a storage device such as an HDD or a flash drive such as an SSD, compact flash (CF) card, SD card, micro-SD card, mini-SD card, Xd card, a memory stick, or the like.

The processing unit 180 may obtain and analyze information related to image content from the image content playback device 110, and may set the massage mode of the massage providing apparatus 100 according to a result of analyzing the information related to image content. Furthermore, the processing unit 180 may analyze the type of image content from the information related to image content, may analyze object movement information including at least one among a movement amount of an object included in the image content and a movement direction of the object, may set a massage course included in the massage mode according to the type of the image content, and may set a massage option included in the massage mode according to the movement information about an arbitrary object included in the image content. Furthermore, the processing unit 180 may obtain at least one among the first history information including a massage mode set by using at least one among the type of image content and the object movement information and the second history information including a massage mode set by receiving a manual operation signal from the user, and may analyze the preference of the user, and may set the massage mode according to a result of analyzing the preference of the user.

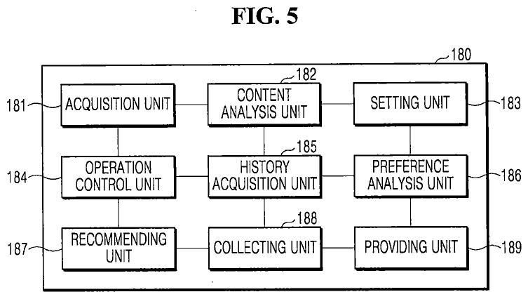

In the present embodiment, the processing unit 180 may be provided outside the control unit 190 as illustrated in FIG. 3, or may be provided in the control unit 190 to operate like the control unit 190, or may be provided in the server 400 of FIG. 1. Detailed operation of the processing unit 180 will be described with reference to FIG. 5.

The control unit 190, which is a type of a central processing unit, may control overall operation of the massage providing apparatus 100 by driving control software installed in the memory 170. In the present embodiment, the control unit 190 may obtain and analyze information related to image content from the image content playback device 110, may set the massage mode of the massage providing apparatus 100 according to a result of analyzing the information related to image content, and may control the massage providing apparatus 100 to operate according to the set massage mode. The control unit 190 may control at least one among a massage driving unit 120, an air adjustment unit 122, a temperature adjustment unit 124, and a main body driving unit 126 according to the set massage mode.

Furthermore, the control unit 190 may analyze the preference of the user on the basis of a manual operation signal of the user and user information detected via the user input unit 160, and may recommend a user-customized massage mode on the basis of a result of analyzing the preference of the user. Furthermore, the control unit 190 may recommend the user-customized massage mode on the basis of the result of analyzing the preference of the user and weather information.

Furthermore, the control unit 190 may further include a voice recognition unit (not shown) so as to control operation of the massage providing apparatus 100 using a speech of the user. To this end, the memory 170 may store a voice recognition algorithm, and may store a start word for driving the massage providing apparatus 100, so that when the user utters the start word, the control unit 190 may operate the voice recognition unit, and the voice recognition unit may recognize the start word to change the massage providing apparatus 100 from a deactivated state to an activated state. After the massage providing apparatus 100 is changed to the activated state, the control unit 190 may recognize a voice command from the user via the sound input unit, and may control operation of the massage providing apparatus 100 according to the voice command. In the present embodiment, the voice recognition unit may be included in the processing unit 180, or may be separately provided outside the control unit 190.

Here, the control unit 190 may include any type of devices capable of processing data, such as a processor. Here, the term “processor” may represent, for example, a hardware-embedded data processing device having a physically structured circuit to execute functions expressed as instructions or codes included in a program. Examples of the hardware-embedded data processing device may include a processing device, such as a microprocessor, a central processing unit (CPU), a processor core, a multiprocessor, an application-specific integrated circuit (ASIC), a field programmable gate array (FPGA), etc., but the scope of the present disclosure is not limited thereto.

In the present embodiment, the control unit 190 may perform analysis of information related to image content (the type of image content, the movement amount of an object included in the image content, the movement direction of the object, etc.), massage mode setting (may include an operation mode other than a massage mode), and machine learning such as deep learning or the like for recommending the user-customized massage mode, and the memory 170 may store data used in the machine learning, result data, etc.

A deep learning technology, which is a type of machine learning, may perform learning to a deep level in stages on the basis of data. The deep learning may represent a set of machine learning algorithms for extracting essential data from a plurality of pieces of data with an increase in stages.

A deep learning structure may include an artificial neural network (ANN), and may be configured with, for example, a deep neural network (DNN) such as a convolutional neural network (CNN), a recurrent neural network (RNN), a deep belief network (DBN), or the like. The deep learning structure according to the present embodiment may use various known structures. For example, the deep learning structure according to the embodiment of the present disclosure may include a CNN, RNN, DBN, or the like. The RNN, which is frequently used for processing natural language, is an efficient structure for processing time-series data that varies with time, and may constitute an artificial neural network by stacking layers up every moment. The DBN may include a deep learning structure configured by stacking multiple layers of restricted Boltzmann machine (RMB) that is a deep learning technique. When the number of layers reaches a certain number by repeating RBM learning, the DBN may have the certain number of layers. The CNN may include a model simulating a human brain function established on the assumption that when recognizing an object, a person extracts basic features of the objects, and then undergoes a complicated calculation in the brain to recognize the object on the basis of a result of the calculation.

Meanwhile, learning of an artificial neural network may be performed by adjusting a weight of a connection line (also adjusting a bias value if necessary) between nodes so that a desired output is achieved with regard to a given input. Furthermore, the artificial neural network may continuously update a weight value through learning. Furthermore, a method of back propagation or the like may be used in the learning of the artificial neural network.

The control unit 190 may include an artificial neural network, for example, a deep neural network (DNN) such as a CNN, RNN, DBN, and the like, and may learn the deep neural network in order to analyze information related to image content, set a massage mode, and recommend a user-customized massage mode. Both unsupervised learning and supervised learning may be used as a machine learning method of the artificial neural network. The control unit 190 may control so as to update an artificial neural network structure after learning according to a setting.

FIG. 5 is a schematic block diagram illustrating the processing unit of the massage providing apparatus of FIG. 3. Descriptions which overlap with the above descriptions related to FIGS. 1 to 4 are not provided below. Referring to FIG. 5, the processing unit 180 may include an acquisition unit 181, a content analysis unit 182, a setting unit 183, an operation control unit 184, a history acquisition unit 185, a preference analysis unit 186, a recommending unit 187, a collecting unit 188, and a providing unit 189.

The acquisition unit 181 may acquire information related to image content from the image content playback device 110. Furthermore, the acquisition unit 181 may acquire the information related to image content about one among augmented reality (AR) image content, virtual reality (VR) image content, and mixed reality (MR) image content. Here, as described above, the information related to image content may include metadata indicating information about the image content and image content identification information. For example, the image content metadata may be information about contents related to image content, such as an image content title, starring actors, a type (genre), etc., and the image content identification information may be unique identification information for discriminating metadata of one or more pieces of content and identifying the one or more pieces of content. That is, the acquisition unit 181 may acquire, from the image content playback device 110, image content and information (image content metadata and image content identification information) related to the image content.

The content analysis unit 182 may analyze the information related to image content acquired by the acquisition unit 181. The content analysis unit 182 may analyze the type of image content from the information related to image content. That is, when the content analysis unit 182 acquires, from the image content playback device 110, the information related to image content, which includes image content type (genre) information, the content analysis unit 182 may directly reflect the acquired type of the image content as an analysis result.

Furthermore, when the content analysis unit 182 acquires, from the image content playback device 110, the information related to image content except for image content type (genre) information, or when the acquired type information about image content is different from information stored in the memory 170 to set the massage mode, the content analysis unit 182 may analyze the type of image content from the information related to image content (image content metadata and image content identification information) on the basis of a classification criterion set in advance in the memory 170 to set the massage mode. For example, when the image content is a movie, the content analysis unit 182 may predict the type of the image content on the basis of the information related to the image content, such as a movie screening period, a movie director, starring actors, a movie title, a movie rating, filming locations, etc., and may classify the image content according to the classification criterion set in advance in the memory 170. This is because different massage modes (massage courses) are required to be set according to the type of image content.

Furthermore, the content analysis unit 182 may analyze object movement information including at least one among a movement amount of an object included in the image content and a movement direction of the object from the information related to the image content. That is, the content analysis unit 182 may select the object included in at least one of frames included in the image content, and may extract a motion vector value (object movement information including at least one among the movement amount and the movement direction) of the object. In other words, the content analysis unit 182 may determine an arbitrary object included in at least one of frames included in the image content, and may extract motion data of the determined object so as to extract the motion vector value of the arbitrary object.

For example, the content analysis unit 182 may obtain a background image in which a moving object has been separated from several consecutive images. Furthermore, the content analysis unit 182 may convert an analog image signal into a digital image signal after obtaining the analog image signal via a frame grabber to divide an image frame to an appropriate size as necessary, and may perform filtering to remove an included noise. Furthermore, the content analysis unit 182 may extract a contour of an image, and then may calculate a location of the moving object through an operation between the contour and a previously binarized and filtered difference of images (obtaining an image by performing a subtraction operation between two images) so as to calculate a more accurate motion vector, and may obtain, from the motion vector, an image of a movement region in an image separated from the background image. Furthermore, the content analysis unit 182 may obtain an object image from an input image by using the input image and information about a contour divided due to separation of the movement region from a background screen, and may extract a feature of the object from the information thereof. This is because different massage modes (massage options) are required to be set according to a motion vector value (object movement information including at least one among the movement amount and the movement direction) of the object of the image content.

The setting unit 183 may set the massage mode of the massage providing apparatus 100 according to a result of analyzing the information related to image content from the content analysis unit 182. According to the related art, one of preset automatic massage modes should be set regardless of image content played back in the image content playback device 110, or the user should manually set the massage mode on the massage mode control panel 141 displayed on the display unit 140. However, in the present embodiment, the setting unit 183 may automatically set the massage mode according to a result of analyzing image content played back in the image content playback device 110 so that convenience may be provided to the user, and a sense of boredom experienced by the user while using a massage chair may be relieved. Furthermore, in the present embodiment, the massage mode is changed according to an image motion vector value of image content to provide an optimal motion suitable for the image content and maximize the effect of a fun function.

The setting unit 183 may set a massage course included in the massage mode according to the type of image content. That is, the setting unit 183 may set the massage course including at least one massage portion among the foot, leg, arm, waist, back, shoulder, neck, head, and whole body of the user sitting on the massage providing apparatus 100. For example, the setting unit 183 may set the massage course to a vitalization course when the type of image content being viewed by the user is a multi-play/social, game, racing, or sports as a result of analyzing the information related to image content, and may set the massage course to a leg/foot course when the type of the image content is a horror/mystery. Furthermore, the setting unit 183 may set the massage course to a neck/shoulder course when the type of the image content being viewed by the user is a world tour or documentary/history, may set the massage course to a back/waist course when the type of the image content is entertainment, may set the massage course to a late-night course when the type of the image content is related to creativity, and may set the massage course to a rest course when the type of the image content is a rest/meditation. Furthermore, the setting unit 183 may set the massage course to a stretching course when the type of the image content being viewed by the user is experienceable, and may set the massage course to a relaxation course when the type of the image content is music.

Here, the massage mode may be roughly divided into an automatic massage mode and a manual massage mode, which may be determined through selection by the user. Furthermore, the massage mode may include a massage course and a massage option, wherein the massage course may represent a type of a massage program obtained by combining massage methods or specific portions such as an acupressure and kneading or a shoulder and waist. Furthermore, the massage option may represent a detailed option of the massage course. A variable option may include, for example, at least one among a massage order, a detailed massage portion, a massage strength, a massage position change direction, a massage direction in the corresponding massage portion, and a movement direction of the massage providing apparatus 100.

Here, in the present embodiment, the setting unit 183 may set the massage option included in the massage mode according to movement information about an arbitrary object included in image content. That is, the setting unit 183 may change and set the massage strength, a massage position change rate (change period), a massage direction change rate in a corresponding massage portion, etc. according to a magnitude of a motion vector of image content. Furthermore, the setting unit 183 may change and set the massage position change direction, the massage direction in a corresponding massage portion, the movement direction of the main body of the massage providing apparatus 100, etc. according to the direction of the motion vector of image content. For example, when the magnitude of the motion vector of image content is calculated as one among 1 to 5, the setting unit 183 may set the massage strength to level 1 to 5 according to the magnitude of the motion vector. Furthermore, the setting unit 183 may change and set the massage course from the leg/foot course to the neck/shoulder course when the direction of the motion vector of image content changes from a downward direction to an upward direction, and may change and set the massage course from the neck/shoulder course to the leg/foot course when the direction of the motion vector of image content changes from the upward direction to the downward direction.

The operation control unit 184 may control the massage providing apparatus 100 to operate according to the massage mode set by the setting unit 183. For example, if the setting unit 183 sets the massage course to the rest course when the type of image content is a rest/meditation, the operation control unit 184 may control operation of the massage driving unit 120, an air adjustment unit 122, the temperature adjustment unit 124, and the main body driving unit 126.

The massage driving unit 120 may drive a massage roller or massage protrusion provided to a user-seated portion of the massage providing apparatus 100 so as to substantially perform a massage such as beating or the like. The air adjustment unit 122 may perform a massage such as kneading or the like through a process of injecting and discharging air. The temperature adjustment unit 124 may adjust a temperature that may be sensed by the user sitting on the massage providing apparatus 100, and the main body driving unit 126 enables the main body of the massage providing apparatus 100 to move according to control by the operation control unit 184. This operation enables the main body of the massage providing apparatus 100 to move according to image content played back in the image content playback device 110 so that the user may be more immersed in the image content being viewed by the user.

The history acquisition unit 185 may obtain at least one among the first history information including a massage mode set by using at least one among the type of image content and the object movement information and the second history information including a massage mode set by receiving a manual operation signal from the user. That is, the history acquisition unit 185 may obtain at least one among a cumulative usage amount, a cumulative usage period, and a cumulative number of times of operation change with regard to the massage modes included in the first history information and second history information. For example, the history acquisition unit 185 may use at least one among the type of image content and the object movement information to obtain history information indicating the number of times an automatically set massage mode has been performed, how many hours the corresponding massage mode has been performed, and the number of times the user has made a change in the automatically set massage mode. Furthermore, when the user manually adjusts using the massage mode control panel 141, the history acquisition unit 185 may obtain a corresponding massage course and massage option.

The preference analysis unit 186 may analyze the preference of the user by using the first history information and the second history information acquired by the history acquisition unit 185. That is, the preference analysis unit 186 may analyze the preference of the user by comparing a reference value with at least one among the cumulative usage amount, the cumulative usage period, and the cumulative number of times of operation change with regard to the massage modes included in the first history information including a massage mode set by using at least one among the type of image content and the object movement information and the second history information including a massage mode set by receiving a manual operation signal from the user. Here, the reference value, which represents a reference value for determining the preference, may be set and stored in the memory 170 in a design stage, and may be changed by the user. For example, the preference analysis unit 186 may analyze how many times the user has used an automatically set massage mode, which item has been manually changed in the automatically set massage mode, how many times the user has used a manually changed massage mode, etc. This is because different massage modes (massage courses and massage options) are required to be set according to the preference of the user so as to reflect a level of satisfaction of an individual user.

Furthermore, the preference analysis unit 186 may analyze the preference of the user by reflecting user information detected via the user input unit 160. That is, the preference analysis unit 186 may more accurately analyze the preference of the user by using the health condition of the user, the emotional state of the user when the user is being massaged, and the like. Therefore, the preference analysis unit 186 may analyze the preference of the user on the basis of at least one among the first history information, the second history information, and the user information detected via the user input unit 160 so as to learn and provide a massage mode further optimized for an individual user. Here, the setting unit 183 may set the massage mode according to a result of analyzing the preference of the user from the preference analysis unit 186, and the operation control unit 184 may operate the massage providing apparatus 100 according to the massage mode set according to the result of analyzing the preference of the user.

The recommending unit 187 may receive the result of analyzing the preference of the user from the preference analysis unit 186 to recommend a user-customized massage mode. When recommending the user-customized massage mode, the recommending unit 187 may combine at least one among the first history information and the second history information stored in the memory 170 and the user information detected via the user input unit 160 to recommend the user-customized massage mode. The recommending unit 187 may learn, for example, a massage mode frequently selected by the user to generate a massage style that the user might like. Furthermore, the recommending unit 187 may recommend the user-customized massage mode according to the type of image content and a real-time state of the user detected via the user input unit 160, on the basis of the massage style that the user might like. That is, for example, when the health condition of the user is not good or the user likes a massage style in which a massage strength is low, the recommending unit 187 may recommend a relatively static massage course even if the type of image content involves fast screen transition. That is, the recommending unit 187, for example, may recommend the rest course or the vitalization course but may change the massage option to a relatively static massage option. Furthermore, the recommending unit 187 may also recommend health-related information together with the user-customized massage mode.

Furthermore, the recommending unit 187 may receive a user's feedback (e.g., like, change, massage execution) for the user-customized massage mode displayed on the display unit 140, and the feedback may be used in learning of the control unit 190.

The collecting unit 188 may receive the weather information from the weather server for providing the weather information. In one exemplary embodiment, the collecting unit 188 may collect the weather information by accessing the server 400, which stores the weather information collected from the weather server. Here, the weather information may include past, current, and future weather (e.g., cloudy, clear, rainy, snowy, etc.), temperature (minimum temperature, maximum temperature, average temperature, etc.), season information (spring, summer, autumn, winter), an air quality index, an ultraviolet index, a humidity index, an aridity index, etc.

The recommending unit 187 may recommend the user-customized massage mode on the basis of the result of analyzing the information related to image content from the content analysis unit 182, the result of analyzing the preference of the user from the preference analysis unit 186, and the weather information collected by the collecting unit 188. For example, the recommending unit 187 may recommend, for a rainy day, the massage mode selected by the user on a past humid day, or may recommend an optimum temperature for a massage according to an air temperature.

Furthermore, the collecting unit 188 may collect health information from a health server for providing the health information. In one exemplary embodiment, the collecting unit 188 may collect the health information by accessing the server 400, which stores the health information collected from the health server. Here, the health information may include health/disease information, symptom/sign information, and examination/treatment information according to age and gender.

The recommending unit 187 may recommend the user-customized massage mode on the basis of the result of analyzing the information related to image content from the content analysis unit 182, the result of analyzing the preference of the user from the preference analysis unit 186, and the health information collected by the collecting unit 188. For example, the recommending unit 187 may recommend the massage mode having a highest massage strength to a male in thirties.

The providing unit 189 may provide health/beauty information, music, news, etc. on the display unit 140. Furthermore, the providing unit 189 may provide a messenger notification or predetermined information so as to play music preferred by the user, or may recognize a speech from the user, for example, “how is the weather today”, so as to provide today weather in response to the speech of the user.

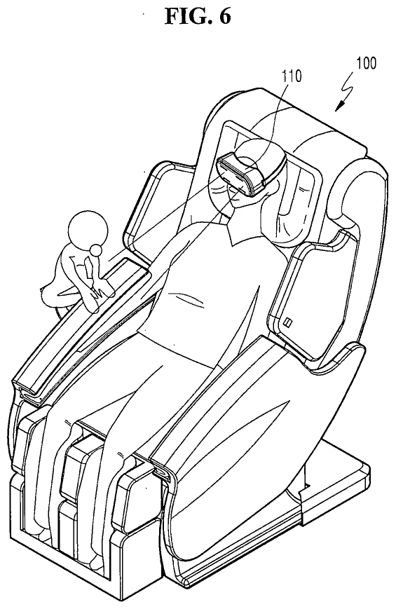

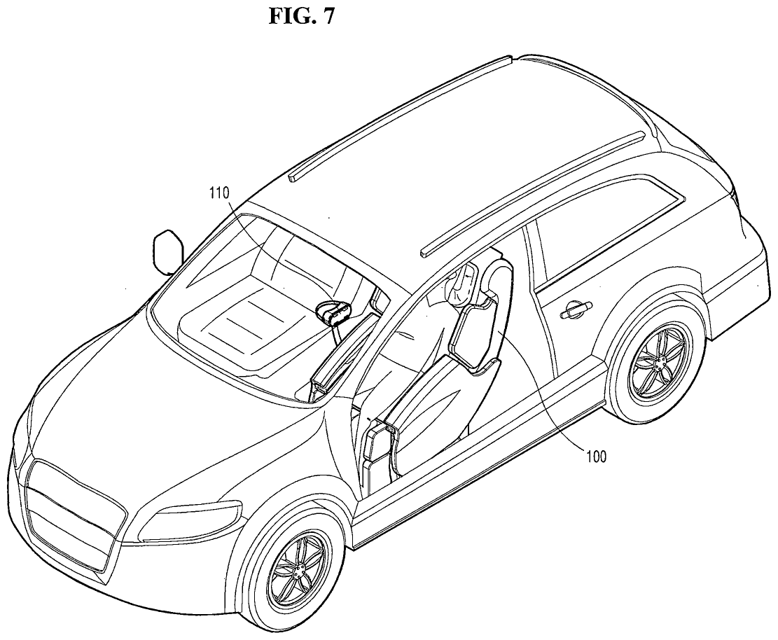

FIG. 6 is an exemplary diagram for describing operation of the massage providing apparatus according to an embodiment of the present disclosure when mixed reality (MR) image content is played back, and FIG. 7 is an exemplary diagram for describing operation of the massage providing apparatus installed in an autonomous vehicle according to an embodiment of the present disclosure. Descriptions which overlap with the above descriptions related to FIGS. 1 to 5 are not provided below.

Referring to FIG. 6, the image content playback device 110 interworking with the massage providing apparatus 100 may play back mixed reality (MR) image content. Here, the mixed reality (MR) may represent an environment in which virtual reality (VR) is mixed with a real world so that an actual physical object may interact with a virtual object. That is, in the present embodiment, when the user who is wearing the image content playback device 110 massage an arm, the image content playback device 110 may synthesize and output image content in which a human-like virtual object (e.g., a 3D graphic character) massages an arm. Accordingly, the user may also be visually provided with a sense of being massaged. Furthermore, in the present embodiment, the image content playback device 110 may synthesize and output image content in which a virtual object acts or answers in response to a speech of the user who is wearing the image content playback device 110. Thus, the fun function of interacting with a virtual object or the like may relieve a sense of boredom experienced by the user during a massage, without affecting an original purpose of receiving a massage and taking a rest.

Referring to FIG. 7, the massage providing apparatus 100 capable of interworking with the image content playback device 110 may be installed in an autonomous vehicle. Here, the massage providing apparatus 100 installed in an autonomous vehicle may be configured by providing a massage function to a seat of an existing vehicle, and may be configured as a typical massage chair type. Meanwhile, when the user receives a massage while wearing the image content playback device 110 in the autonomous vehicle, the image content playback device 110 may play back virtual reality (VR) image content, but it may be desirable to play back mixed reality (MR) image content since the autonomous vehicle is travelling.

In an optional embodiment, the massage providing apparatus 100 may recommend the massage mode on the basis of travel information and traffic information. To this end, the massage providing apparatus 100 may collect the travel information from the vehicle, and may collect the traffic information from a traffic server (not shown) for providing the traffic information. Here, the massage providing apparatus 100 may communicate with the traffic server via the network 500. That is, the massage providing apparatus 100 may combine at least one among the image content analysis result, the user preference, the travel information, and the traffic information so as to recommend and set the massage mode. For example, even if the same image content is provided, a massage course having a higher massage strength may be recommended and set when a traffic flow is smooth compared to when traffic is congested.

In the present embodiment, the massage providing apparatus 100 may be provided in the autonomous vehicle, and may search for and select at least one output apparatus 200, which is disposed in the same space as the massage providing apparatus 100 to output at least one among an audio signal and a video signal. Here, the output apparatus 200 may include an electronic device for outputting at least one among an audio signal and a video signal, for example, an in-vehicle speaker (not shown), the AI speaker 230, and an AVN device (not shown) such as a navigation device and a head-up display (HUD). Furthermore, in the present embodiment, the AVN device such as a navigation device and a head-up display (HUD) may be the display unit 140.

Meanwhile, in the present embodiment, the collecting unit 188 may collect the travel information from the vehicle, and may collect the traffic information from the traffic server for providing the traffic information. Here, the travel information may include a vehicle travel speed, travel mode, route guide information (e.g., remaining time to destination), and the like. Furthermore, the traffic information may include traffic situation information, information about an external environment in which the vehicle is travelling, and the like. Here, the information about an external environment in which the vehicle is travelling may include not only information about a road (expressway, national highway, general road, etc.) along which the vehicle is travelling but also weather information (weather, air temperature, season information, air quality index, ultraviolet index, humidity index, aridity index, etc.).

The recommending unit 187 may recommend the user-customized massage mode on the basis of the result of analyzing the information related to image content from the content analysis unit 182, the result of analyzing the preference of the user from the preference analysis unit 186, and the travel information and the traffic information collected by the collecting unit 188. For example, when traffic congestion occurs, the recommending unit 187 may recommend the rest course to provide a comfortable massage, or may rather recommend the vitalization course to relieve a sense of boredom due to the traffic congestion. That is, the recommending unit 187 may combine at least one among the result of analyzing the information related to image content, the result of analyzing the preference of the user from the preference analysis unit 186, and the travel information and the traffic information collected by the collecting unit 188 so as to recommend the user-customized massage mode.

Furthermore, in the present embodiment, when the user is wearing the image content playback device 110 in the autonomous vehicle, an actual travel screen and image content (particularly a virtual object) may be synthesized and output. Here, the massage providing apparatus 100 may change and set the massage mode according to an actual travel situation. Furthermore, in the present embodiment, the image content playback device 110 may synthesize and output image content in which a virtual object acts or answers in response to a speech of the user who is wearing the image content playback device 110. Therefore, in the present embodiment, the massage mode which is automatically set according to the actual travel situation may be provided, and the fun function of interacting with a virtual object or the like may be provided so that a sense of boredom may be relived during a massage, and the travel situation may also be checked, and the user may take a rest while receiving a massage.

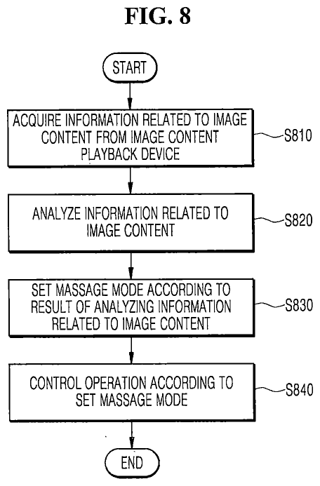

FIG. 8 is a flowchart illustrating a method for operating a massage providing apparatus according to an embodiment of the present disclosure. Descriptions which overlap with the above descriptions related to FIGS. 1 to 7 are not provided below.

Referring to FIG. 8, in operation S810, the massage providing apparatus 100 acquires the information related to image content from the image content playback device 110. The massage providing apparatus 100 may acquire, from the image content playback device 110, the information related to image content about one among augmented reality (AR) image content, virtual reality (VR) image content, and mixed reality (MR) image content. Here, the information related to image content may include metadata indicating information about the image content and image content identification information. That is, the massage providing apparatus 100 may acquire, from the image content playback device 110, image content and information (image content metadata and image content identification information) related to the image content.

In operation S820, the massage providing apparatus 100 analyzes the acquired information related to image content. When the massage providing apparatus 100 acquires, from the image content playback device 110, the information related to image content, which includes image content type (genre) information, the massage providing apparatus 100 may directly reflect the acquired type of the image content as an analysis result. Furthermore, when the massage providing apparatus 100 acquires, from the image content playback device 110, the information related to image content except for image content type (genre) information, the massage providing apparatus 100 may analyze the type of image content on the basis of a classification criterion set in advance in the memory 170 to set the massage mode. For example, when the image content is a movie, the massage providing apparatus 100 may predict the type of the image content on the basis of the information related to the image content, such as a movie screening period, a movie director, starring actors, a movie title, a movie rating, filming locations, etc., and may classify the image content according to the classification criterion set in advance in the memory 170. This is because different massage modes (massage courses) are required to be set according to the type of image content. Furthermore, the massage providing apparatus 100 may analyze object movement information including at least one among a movement amount of an object included in the image content and a movement direction of the object from the information related to the image content. That is, the massage providing apparatus 100 may select the object included in at least one of frames included in the image content, and may extract a motion vector value (object movement information including at least one among the movement amount and the movement direction) of the object. This is because different massage modes (massage options) are required to be set according to a motion vector value (object movement information including at least one among the movement amount and the movement direction) of the object of the image content.

In operation S830, the massage providing apparatus 100 sets the massage mode according to the result of analyzing the information related to image content. The massage providing apparatus 100 may set a massage course included in the massage mode according to the type of image content. That is, the massage providing apparatus 100 may set the massage course including at least one massage portion among the foot, leg, arm, waist, back, shoulder, neck, head, and whole body of the user sitting on the massage providing apparatus 100. Furthermore, the massage providing apparatus 100 may set the massage option included in the massage mode according to movement information about an arbitrary object included in image content. That is, the massage providing apparatus 100 may change and set the massage strength, the massage position change rate (change period), the massage direction change rate in a corresponding massage portion, etc. according to the magnitude of a motion vector of image content. Furthermore, the massage providing apparatus 100 may change and set the massage position change direction, the massage direction in a corresponding massage portion, the movement direction of the main body of the massage providing apparatus 100, etc. according to the direction of the motion vector of image content.

In operation S840, the massage providing apparatus 100 controls operation according to the set massage mode. For example, if the massage providing apparatus 100 sets the massage course to the rest course when the type of image content is a rest/meditation, the massage providing apparatus 100 may control operation of the massage driving unit 120, the air adjustment unit 122, the temperature adjustment unit 124, and the main body driving unit 126.

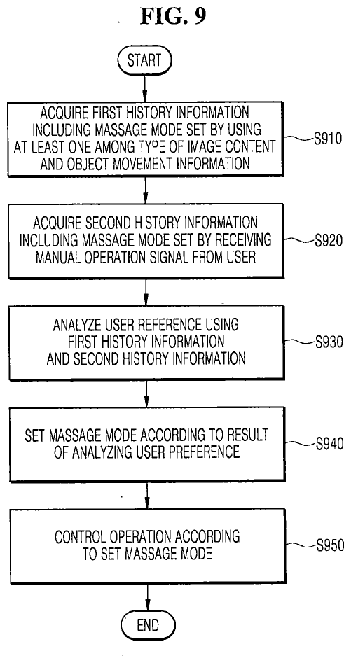

FIG. 9 is a flowchart illustrating a method for operating a massage providing apparatus by reflecting a result of analyzing the preference of a user according to an embodiment of the present disclosure. Descriptions which overlap with the above descriptions related to FIGS. 1 to 8 are not provided below.

Referring to FIG. 9, in operation S910, the massage providing apparatus 100 acquires the first history information including the massage mode set by using at least one among the type of image content and the object movement information. For example, the massage providing apparatus 100 may use at least one among the type of image content and the object movement information to obtain history information indicating the number of times an automatically set massage mode has been performed, how many hours the corresponding massage mode has been performed, and the number of times the user has made a change in the automatically set massage mode.

In operation S920, the massage providing apparatus 100 acquires the second history information including the massage mode set by receiving a manual operation signal from the user. That is, when the user manually operates using the massage mode control panel 141, the massage providing apparatus 100 may obtain a corresponding massage course and massage option. That is, in operation S910 and operation S920, the massage providing apparatus 100 may obtain at least one among a cumulative usage amount, a cumulative usage period, and a cumulative number of times of operation change with regard to the massage modes included in the first history information and second history information.

In operation S930, the massage providing apparatus 100 analyzes the preference of the user by using the acquired first history information and second history information. That is, the massage providing apparatus 100 may analyze the preference of the user by comparing a reference value with at least one among the cumulative usage amount, the cumulative usage period, and the cumulative number of times of operation change with regard to the massage modes included in the first history information including the massage mode set by using at least one among the type of image content and the object movement information and the second history information including the massage mode set by receiving a manual operation signal from the user. This is because different massage modes (massage courses and massage options) are required to be set according to the preference of the user so as to reflect a level of satisfaction of an individual user.

Furthermore, for example, the massage providing apparatus 100 may analyze the preference of the user by reflecting the user information detected via the user input unit 160. That is, the massage providing apparatus 100 may more accurately analyze the preference of the user by using the health condition of the user, the emotional state of the user when the user is being massaged, and the like. Therefore, the massage providing apparatus 100 may analyze the preference of the user on the basis of at least one among the first history information, the second history information, and the user information detected via the user input unit 160 so as to learn and provide a massage mode further optimized for an individual user.

In operation S940, the massage providing apparatus 100 sets the massage mode according to the result of analyzing the preference of the user. Meanwhile, the massage providing apparatus 100 may receive the result of analyzing the preference of the user to recommend and set the user-customized massage mode. When recommending and setting the user-customized massage mode, the massage providing apparatus 100 may combine at least one among the first history information and the second history information stored in the memory 170 and the user information detected via the user input unit 160 to recommend and set the user-customized massage mode. Furthermore, the massage providing apparatus 100 may learn, for example, a massage mode frequently selected by the user to generate a massage style that the user might like. Furthermore, the massage providing apparatus 100 may recommend and set the user-customized massage mode according to the type of image content and a real-time state of the user detected via the user input unit 160, on the basis of the massage style that the user might like.

In operation S950, the massage providing apparatus 100 operates the massage providing apparatus 100 according to the massage mode set according to the result of analyzing the preference of the user. That is, the massage providing apparatus 100 may drive a massage roller or massage protrusion provided to a user-seated portion of the massage providing apparatus 100 via the massage driving unit 120 so as to substantially perform a massage such as beating or the like. Furthermore, the massage providing apparatus 100 may perform a massage such as kneading or the like through a process of injecting and discharging air via the air adjustment unit 122.

According to an embodiment of the present disclosure, a massage mode of a massage providing apparatus may be automatically set and operated on the basis of information related to image content of an image content playback device so as to improve user's satisfaction with the use of the massage providing apparatus and relieve a sense of boredom experienced by the user while using the massage providing apparatus.

Furthermore, user's satisfaction may be improved and the effect of a fun function may be maximized by changing detailed options of a massage mode on the basis of object movement information including at least one among a movement amount of an object included in image content and a movement direction of the object and performing a motion control optimized for the image content.

Furthermore, a further optimized massage mode may be provided to a user to improve user's satisfaction with the use of a massage providing apparatus by recommending and setting a massage mode in which user's preference is reflected on the basis of first history information based on information related to image content and second history information based on a manual operation signal from the user.

Furthermore, user's satisfaction may be improved by recommending a user-customized massage mode on the basis of information related to image content, user preference, and weather information.

Furthermore, user's satisfaction may be improved by recommending a user-customized massage mode on the basis of information related to image content, user preference, weather information, and health information.

Furthermore, convenience of a user may be improved by supporting a function of charging an image content playback device in a massage providing apparatus.

Furthermore, user's satisfaction may be improved by visually providing a sense of being massaged by synthesizing and outputting image content including a virtual massaging object to a real world on the basis of mixed reality (MR) image content.

Furthermore, a sense of boredom experienced by a user may be relieved during a massage and the original purpose of receiving a massage and taking a rest may be satisfied by synthesizing and outputting image content including a virtual massaging object to a real world on the basis of mixed reality (MR) image content and by providing a function in which the virtual objects acts or dialogizes in response to a speech of the user.

Furthermore, a massage mode of a massage providing apparatus may be automatically set and operated on the basis of at least one among information related to image content, user preference, travel information, traffic information, health information, and weather information in an autonomous vehicle, so as to improve user's satisfaction with the use of the massage providing apparatus and improve the usability of the massage providing apparatus.

Although a massage providing apparatus is a product that is mass produced and uniform, a user recognizes a massage providing apparatus as a personalized apparatus, and thus an embodiment of the present disclosure may bring about the effect of providing a user-customized product.

The effects of the present disclosure are not limited to the above-mentioned effects, and other effects would be clearly understood by those skilled in the art from the following description.

The above-mentioned embodiments of the present disclosure may be implemented as a computer program executable by various components on a computer, and such the computer program may be recorded in a computer-readable medium. Here, the medium may include magnetic media such as hard discs, floppy discs, and magnetic tapes, optical media such as CD-ROM and DVD, magneto-optical media such as floptical disc, and hardware devices specially configured to store and perform program codes, such as ROM, RAM, and flash memory.

The computer program may be one specially designed and configured for the present disclosure, or may be one well known and available in the technical fields of computer software. Examples of the computer program may include not only machine language codes generated by compilers but also high-level language codes that can be executed by computers using interpreters.

The singular forms “a,” “an” and “the” in this present disclosure, in particular, claims, may be intended to include the plural forms as well. Unless otherwise defined, the ranges defined herein is intended to include any invention to which values within the range are individually applied and may be considered to be the same as individual values constituting the range in the detailed description of the present invention.

Operations constituting the methods of the present disclosure may be performed in appropriate order unless explicitly described in terms of order or described to the contrary. The present disclosure is not necessarily limited to the order of operations given in the description. The examples or exemplary terms (for example, “or the like”, “and the like”, “etc.”) used herein are to merely describe the present invention in detail and not intended to limit the present invention unless defined by the following claims. Furthermore, those skilled in the art will readily appreciate that many alternation, combination and modifications, may be made according to design conditions and factors within the scope of the appended claims and their equivalents.

Therefore, the scope of the present disclosure is defined not by the detailed description, but by the claims and their equivalents, and all variations within the scope of the claims and their equivalents are to be construed as being included in the present disclosure.

What is claimed is:

1. A method for controlling a massage providing apparatus configured to interwork with an image content playback device, the method comprising:
acquiring, by a controller, information related to image content from the image content playback device;
analyzing the information related to the image content;
setting a massage mode according to a result of analyzing the information related to the image content; and
controlling the massage providing apparatus to operate according to the set massage mode.

2. The method of claim 1, wherein analyzing the information related to the image content comprises:
analyzing a type of the image content from the information related to the image content; and
analyzing object movement information comprising at least one of a movement amount of an object included in the image content or a movement direction of the object included in the image content from the information related to the image content.

3. The method of claim 2, wherein setting the massage mode comprises setting a massage course included in the massage mode according to the type of the image content.

4. The method of claim 3, wherein the massage course comprises at least one massage portion of a foot, a leg, an arm, a waist, a back, a shoulder, a neck, a head, or a whole body of a user sitting on the massage providing apparatus.

5. The method of claim 2, wherein setting the massage mode comprises setting a massage option included in the massage mode according to the object movement information.

6. The method of claim 5, wherein the massage option comprises at least one of a massage strength of the massage providing apparatus, a massage position change direction, a massage direction in a corresponding massage portion, or a movement direction of the massage providing apparatus.

7. The method of claim 2, further comprising:
acquiring at least one of first history information comprising a previous massage mode set by using the at least one of the type of the image content and the object movement information or second history information comprising a previous massage mode set by receiving a manual operation signal from a user;
analyzing a preference of the user using at least one of the first history information or the second history information; and
updating the massage mode according to a result of analyzing the preference of the user.

8. The method of claim 7, wherein acquiring the at least one of the first or second history information further comprises acquiring at least one of a cumulative usage amount, a cumulative usage period, and a cumulative number of times of operation change with regard to the previous massage modes included in the first history information or the second history information.

9. The method of claim 1, wherein the image content from the image content playback device includes at least one of augmented reality (AR) image content, virtual reality (VR) image content, and mixed reality (MR) image content.

10. A computer program recorded in a computer-readable medium to execute the method of claim 1 using a computer.

11. A massage providing apparatus capable of interworking with an image content playback device, the massage providing apparatus comprising:
an information acquirer configured to acquire information related to image content from the image content playback device;
a content analyzer configured to analyze the information related to the image content;
a massage mode setter configured to set a massage mode according to a result of analyzing the information related to the image content; and
a controller configured to control the massage providing apparatus to operate according to the set massage mode.

12. The massage providing apparatus of claim 11, wherein the content analyzer is configured to:
analyze a type of the image content from the information related to the image content; and
analyze object movement information comprising at least one of a movement amount of an object included in the image content or a movement direction of the object included in the image content from the information related to the image content.

13. The massage providing apparatus of claim 12, wherein the massage mode setter is configured to set a massage course included in the massage mode according to the type of the image content.

14. The massage providing apparatus of claim 13, wherein the massage course comprises at least one massage portion of a foot, a leg, an arm, a waist, a back, a shoulder, a neck, a head, or a whole body of a user sitting on the massage providing apparatus.

15. The massage providing apparatus of claim 12, wherein the massage mode setter is configured to set a massage option included in the massage mode according to the object movement information.

16. The massage providing apparatus of claim 15, wherein the massage option comprises at least one of a massage strength of the massage providing apparatus, a massage position change direction, a massage direction in a corresponding massage portion, and a movement direction of the massage providing apparatus.

17. The massage providing apparatus of claim 12, further comprising:
a history acquirer configured to:
acquire at least one of first history information comprising a previous massage mode set by using the at least one of the type of the image content or the object movement information; or
acquire second history information comprising a previous massage mode set by receiving a manual operation signal from a user; and
a reference analyzer configured to analyze a preference of the user using at least one of the first history information or the second history information,
wherein the massage mode setter is configured to update the massage mode according to a result of analyzing the preference of the user.

18. The massage providing apparatus of claim 17, wherein the history acquirer is configured to further acquire at least one of a cumulative usage amount, a cumulative usage period, and a cumulative number of times of operation change with regard to the previous massage modes included in the first history information and the second history information.

19. The massage providing apparatus of claim 11, wherein the image content from the image content playback device includes at least one of augmented reality (AR) image content, virtual reality (VR) image content, or mixed reality (MR) image content.

20. The massage providing apparatus of claim 11, wherein the massage providing apparatus is connected to the image content playback device via at least one of a cradle, a wired cable, or a wireless communicator.
